(19) United States
(12) Patent Application Publication
(10) Pub. No.: US 2004/0028616 A1
(43) Pub. Date: Feb. 12, 2004

(54) Inhibition of lung metastases by aerosol delivery of p53 gene and anti-cancer compounds

(76) Inventors: J. Vernon Knight, Houston, TX (US); Brian Gilbert, Houston, TX (US); Nadezhda Koshkina, Houston, TX (US); L. Clifford Waldrep, The Woodlands, TX (US); Charles L. Densmore, The Woodlands, TX (US); Ajay Gautam, Houston, TX (US)

Correspondence Address: Benjamin Aaron Adler, ADLER & ASSOCIATES, 8011 Candle Lane, Houston, TX, 77071, US

(21) Appl. No.: 10/366,937

(22) Filed: Feb. 14, 2003

Related U.S. Application Data
(60) Provisional application No. 60/356,864, filed on Feb. 14, 2002.

(51) Int. Cl. A61K 48/00; A61L 9/04; A61K 9/127

(52) U.S. Cl. 424/45; 514/44; 424/450

(57) ABSTRACT

The present invention provides a method of inhibiting growth of lung metastases in an individual comprising the steps of administering in a combination an aerosolized polyethylenimine-DNA complex and an aerosolized liposome-anticancer drug complex with both of the complexes delivered via aerosolization. Delivery of both the DNA and the anticancer drug via this method inhibits growth of lung metastases in the individual. Also provided is a method of inhibiting growth of lung metastases in an individual by the administration in combination via aerosolization of a polyethylenimine-p53 complex and a dilauroylphosphatidylcholine-9-nitrocamptothecin complex.

### CROSS-REFERENCE TO RELATED APPLICATIONS

This non-provisional application claims priority of provisional application U.S. S. No. 60/356,864, filed Feb. 14, 2002, now abandoned.

### BACKGROUND OF THE INVENTION

1. Field of the Invention

This invention relates to the fields of pharmacology and cancer treatment. More specifically, this invention relates to the sequential aerosol delivery of the p53 gene and anti-cancer compounds as an effective method for the growth inhibition of lung metastatic tumors.

2. Description of the Related Art

Lung cancer is the single largest cause of cancer deaths with less than 15% of newly diagnosed patients surviving beyond 5 years. More than 80% of lung cancers do not respond favorably to chemotherapy. Treatment of established lung metastases is a difficult challenge in clinical settings, and often requires multi-modality approaches. One of the primary clinical challenges is to overcome the tumor resistance to chemotherapeutic drugs. P53 tumor suppressor gene mutations are reported in a majority of lung cancers and loss of p53 function results in increased drug resistance and tumor relapse (1). An approach postulated in recent years is p53 gene replacement into the tumor cells that can then lead the cell into apoptosis as well as increase the sensitivity of the cells to chemotherapeutic drugs (2,3).

Most anti-cancer drugs have been conventionally delivered via oral or intravenous routes. Biodistribution of the drugs through these delivery strategies is widespread, with the ratio of the drug deposited in the lungs being low (4). A similar distribution of transgene expression in various tissues is observed after intravenous or intraperitonial delivery of vector-DNA complexes for gene therapy (5). Another concern is the systemic toxicity observed after oral or intravenous routes of delivery. Aerosol delivery of genes and chemotherapeutic drugs represents a very promising technology to target the lungs specifically, increasing the pulmonary deposition and pharmacokinetic profile of the drugs and genes (4,6).

Polyethylenimine (PEI) can achieve high levels of transgene expression in the lungs after aerosol delivery (6), with minimal toxicity or cytokine responses (6,7). The dose of polyethylenimine delivered by aerosol has been shown to result in a n anti-tumor effect in two different lung cancer models using the p53 tumor suppressor gene (8,9). Aerosol delivery of polyethyleniminep53 complexes leads to some evidence of apoptosis in the B16-F10 tumor foci in the lung. Immunohistochemistry for transgene expression shows that polyethylenimine-DNA complexes transfect the B16-F10 tumor foci in the lungs (13).

Aerosol delivery of a liposomal formulation of 9-nitrocamptothecin (9NC), a topoisomerase I inhibitor, such as a dilauroylphosphatidylcholine liposome formulation of 9-nitrocamptothecin (9NC-DLPC) can inhibit the growth of subcutaneous tumors as well as lung metastases (10,11). However, although both the p53 gene and the camptothecins, including 9-nitrocamptothecin, can inhibit subcutaneous tumor growth, as well as lung metastases, alone and in combination with other agents (8,10,11,14,15), the dose delivered is high. Aerosol treatment with 9-nitrocamptothecin requires 5 mg 9-nitrocamptothecin delivered to mice twice daily 5 days a week and the treatment was started on day 1 after tumor inoculation. The dosage of 9-nitrocamptothecin may be varied from about 1 to 9 mg/day (28). Similarly, aerosol delivery of PEI-p53 DNA complexes lead to a significant inhibition of B16-F10 pulmonary metastases when the treatment was started on day 1 after tumor inoculation with a twice a week regimen (8); gene therapy was inactive if treatment started when micrometastases were already established in the lung.

Camptothecins are topoisomerase inhibitors that induce apoptosis in tumor cells by inducing single strand breaks in the DNA (16). P53 has been shown to sensitize various tumor cell lines to camptothecins, although camptothecins and analogs induce apoptosis in some cells lines via a p53 independent pathway (17,18). Also, Kalechman et. al., (19) have previously shown that AS101 (ammonium trichloro(dioxoethylene-0,0′)tellurate), can increase apoptosis in B16-F10 cells via up regulation of p53.

Angiogenesis is required for a tumor to grow beyond 1-2 mm in diameter (20). Additionally, camptothecins have been shown to inhibit tumor growth through modulation of angiogenesis by damaging the actively growing new blood vessels in the tumors (21,22). P53 has been shown to be a key regulator of angiogenesis by transcriptional control of various angiogenic factors such as vascular endothelial growth factor (VEGF) and anti-angiogenic factors such as thrombospondin-1 (TSP-1) (23,24). The B16-F10 cell line is highly angiogenic in vivo, and p53 transfection of B16-F10 cells can reverse this angiogenic phenotype and inhibit tumor growth through down regulation of vascular endothelial growth factor and up regulation of TSP-1 (13).

Studies have demonstrated that p53 can sensitize the tumors to anti-cancer agents in animal models (25) and some of these combination studies are also being incorporated into initial phases of clinical trials (26). Fujiwara et al have demonstrated that p53 can sensitize tumor cells to the DNA damaging drug, cisplatinum both in vitro and in vivo (25). A modest clinical benefit was also demonstrated using this approach in clinical trials (26). The p53 gene in these studies was delivered using cationic liposomes or adenoviruses. Moreover, the technique used for administering gene delivery vectors to lung cancer patients is mainly through intratumoral injections by use of computed tomography-guided percutaneous fine-needle injections or bronchoscopy (26,27). Although showing some therapeutic response, these techniques are rather invasive.

Since most of the gene transfection is localized in the bronchial epithelium (13), a direct effect of p53 expression is highly likely for the tumors of bronchial and alveolar epithelium with a potential indirect effect on tumor growth in any of the pulmonary sites. Aerosol delivery of 9-nitrocamptothecin would further have an added inhibitory effect on tumor growth. Given that p53 can sensitize tumor cells to the action of camptothecins and, furthermore, given the role of both camptothecins and the p53 gene in tumor suppression by regulating anti-angiogenic factors, it is contemplated that combining p53 and 9-nitrocamptothecin would arrest growth of established lung metastases.

The inventors have recognized a need in the art for a n improved method of therapy for established lung metastatic tumors using sequential aerosol delivery of gene/anti-cancer drug combinations. The prior art is deficient in the lack of a method for using the p53 gene sequentially with anti-cancer drugs to inhibit growth of pulmonary metastatic tumors. The present invention fulfills this longstanding need and desire in the art.

### SUMMARY OF THE INVENTION

In one embodiment of the present invention, there is provided a method of inhibiting growth of lung metastases in a n individual comprising the steps of administering in a combination an aerosolized polyethylenimine-nucleic acid complex and an aerosolized liposome-anticancer drug complex such that delivery of both nucleic acid and the anticancer drug inhibits growth of lung metastases in said individual.

In another embodiment of the present invention there is provided a method of inhibiting growth of lung metastases in a n individual comprising the steps of administering in a combination an aerosolized polyethylenimine-p53 complex and an aerosolized dilauroylphosphatidylcholine-9-nitrocamptothecin complex such that delivery of both p53 and 9-nitrocamptothecin inhibits growth of lung metastases in the individual.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention are briefly summarized. Details of the above may be had by reference to certain embodiments thereof which are illustrated in the appended drawings. These drawings form a part of the specification. It is to be noted; however, that the appended drawings illustrate preferred embodiments of the invention and therefore are not to be considered limiting in their scope.

FIG. 1 depicts the effect of p53 gene and 9-nitrocamptothecin on proliferation of B16-F10 cells in vitro.

FIG. 2 depicts the effect of p53 and 9-nitrocamptothecin combination on growth of established B16-F10 lung metastases.

FIG. 2A compares the tumor index to control and PEI:p53- and/or 9-nitrocamptothecin-treated B16-F10 lung metastases. Values are mean+SD (n=10 mice per group).

FIG. 2B depicts representative lungs from untreated (top row), p53 (middle row), and 9-nitrocamptothecin and p53 combination (bottom row) treated mice (n=10 mice per group).

FIG. 2C depicts representative lungs from untreated (top), 9-nitrocamptothecin and Neobam (middle) and 9-nitrocamptothecin and p53 (bottom) combination treated mice are presented (n=10 mice per group). Data is representative of at least 2 separate experiments.

FIG. 2D depicts lung weights of mice from different groups. Values are mean+SD (n=10 mice per group).

FIG. 3 depicts survival time of mice (n=10 mice per group) treated with PEI-p53 aerosol complexes. Data is representative of at least 2 separate experiments (p<0.05; Mann-Whitney rank sum test).

FIG. 4 compares the tumor size and number of tumor foci in control and PEI:p53- and/or PTX-treated LM-6 osteosarcoma pulmonary metastases.

### DETAILED DESCRIPTION OF THE INVENTION

In one embodiment of the present invention, there is provided a method of inhibiting growth of lung metastases in a n individual comprising the steps of administering in a combination an aerosolized polyethylenimine-nucleic acid complex and an aerosolized liposome-anticancer drug complex such that delivery of both nucleic acid and the anticancer drug inhibits growth of lung metastases in said individual.

In one aspect of this embodiment the aerosolized polyethylenimine-nucleic acid complex and an aerosolized liposome-anticancer drug complex are administered simultaneously. In another aspect of this embodiment the aerosolized polyethylenimine-nucleic acid and an aerosolized liposome-anticancer drug complex are administered sequentially. Further to this aspect the aerosolized liposome-anticancer drug complex may be repeatedly administered. In all of these aspects the combination used to administer the complexes may be repeated at least once.

In all aspects of this embodiment, a ratio of polyethylenimine nitrogen to nucleic acid phosphate, or nitrogen:phosphate ratio, of about 2:1 to about 50:1, preferably about 5:1 to about 20:1, can be used. The nucleic acid may be DNA, RNA, a catalytically active nucleic acid, a ribozyme, an antisense oligonucleotide, or a modified nucleic acid. The nucleic acid may exhibit tumor suppressor activity. Representative examples of DNA which may be used in the polyethylenimine-nucleic acid complex in this embodiment are the p53 gene, a truncated derivative of p53, CD1 gene, IL-12 and INF-γ. The liposome-anticancer drug complex may comprise 9-nitrocamptothecin, paclitaxel, doxorubicin, carboplatin, methotrexate, vinblastine, etoposide, docetaxel hydroxyurea, fluorouracil, busulfan, imatinib mesylate, alembuzumab, aldesleukin, and cyclophosphamide.

The polyethylenimine-nucleic acid complex may be a polyethylenimine-p53 complex containing about 2 mg p53/10 ml of aerosol having a nitrogen:phosphate ratio of 5:1 to 20:1, preferably 10:1. Also, the liposome-anticancer drug is a DLPC-9-nitrocamptothecin liposome containing about 0.5 mg 9-nitrocamptothecin/ml at a 9-nitrocamptothecin:DLPC weight ratio of about 1:50. Additionally, the polyethylenimine-nucleic acid a n d liposome-anticancer drug complexes may be administered in an aerosol comprising about 3% to about 7.5% carbon dioxide.

In another embodiment of the present invention there is provided a method of inhibiting growth of lung metastases in a n individual comprising the steps of administering in a combination an aerosolized polyethylenimine-p53 complex and an aerosolized dilauroylphosphatidylcholine-9-nitrocamptothecin complex such that delivery of both p53 and 9-nitrocamptothecin inhibits growth of lung metastases in the individual. The dosages and composition of the polyethylenimine-p53 complex and the dilauroylphosphatidylcholine-9NC complex and the means of administering are as disclosed supra.

The following definitions are given for the purpose of facilitating understanding of the inventions disclosed herein. Any terms not specifically defined should be interpreted according to the common meaning of the term in the art.

As used herein, the term “individual” shall refer to animals and humans.

Provided herein is a method of inhibiting growth of lung metastases in an individual via aerosol delivery of PEI-nucleic acid and liposomal anti-cancer drug complexes. The in vivo model closely replicates a clinical situation where patients present with established pulmonary tumors and treatment of such a disease often requires a multi-modality intervention. Generally, polyethylenimine is used to complex with a nucleic acid such as DNA from genes having tumor suppressor activity and can be delivered in a combinatorial regimen with a liposomal anti-cancer drug composition, e.g., 9-nitrocamptothecin, paclitaxel, doxorubicin, carboplatin, either sequentially or simultaneously. Furthermore, either of the nucleic acid or anti-cancer drug may be administered more than once in different combinations.

Anti-cancer drugs further may encompass, inter alia, antineoplastic agents such as antimetabolites, alkylating agents, Bcr-Abl tyrosinase inhibitors, interleukin-2-derivitase, nitrogen mustard derivatives and folic acid antagonists. Without being limiting examples of such agents are docetaxel hydroxyurea, vinblastine or etoposide, fluorouracil or busulfan, imatinib mesylate or alembuzumab, aldesleukin, cyclophosphamide, and methotrexate, respectively.

Particularly PEI-p53 and 9-nitrocamptothecin-dilauroylphosphatidylcholine aerosol complexes can achieve an enhanced therapeutic response against a B16-F10 melanoma challenge as compared to either agent alone. The lung weights and tumor burden are significantly reduced in mice treated with both p53 and 9-nitrocamptothecin as compared to controls or either p53 or 9-nitrocamptothecin singly.

Additionally, a much lower dose, i.e., about at least a two fold reduction, of p53 and 9-nitrocamptothecin is required to inhibit the growth of established B16-F10 lung metastases when delivered in sequence, even though neither the p53 nor the 9-nitrocamptothecin alone is potent enough to suppress metastatic growth or increase the survival of tumor bearing mice. The dosage regimen found to be effective encompasses the delivery of PEI-p53 before treatment with 9-nitrocamptothecin-dilauroylphosphatidylcholine, rather than drug before gene. It is contemplated that delivery of liposomal drug formulations results in decreased transgene expression levels and a refractory period for PEI-DNA aerosol delivery. The sequential aerosol delivery of p53 and 9-nitrocamptothecin leads to a 30-40% increase in the mean survival time of the B16-F10 melanoma challenged mice, as compared to those animals in different control groups. Also, the aerosol delivery of the drug with about 3%-7.5% CO₂ can lead to increased pulmonary deposition and pharmacokinetics of the 9-nitrocamptothecin.

It is contemplated that other nucleic acids having tumor suppressor activity can be used in combination with the anti-cancer compounds disclosed herein. PEI may be complexed with, for example, other DNA, RNA, a catalytically active nucleic acid, a ribozyme, an antisense oligonucleotide, or a modified nucleic acid. Without being limiting such genes as the CD1 gene, a truncated derivative of p53, 11-12 and INF-γ have demonstrated anticancer potential in vivo. Additionally, the role of anti-angiogenic factors regulated by p53 in tumor suppression could indirectly effect tumor growth in any of the pulmonary sites, even possibly for p53 positive tumors such as small cell lung carcinoma (SCLC).

The following examples are given for the purpose of illustrating various embodiments of the invention and are not meant to limit the present invention in any fashion.

### EXAMPLE 1

Preparation of PEI-p53 Complexes and 9-NC Liposomes

PEI-DNA complexes were prepared as described in ref 6. Neobam (Neo) is the plasmid backbone of the p53 gene containing plasmid, but without the p53 gene. P53 complexes are prepared at a N:P ratio of 5:1; the polyethylenimine nitrogen:DNA phosphate ratio can be calculated by taking into account that 1 μg of DNA has 3 nmol of phosphate and 1 μl of 0.1M polyethylenimine solution has 100 nmol of amine nitrogen, and 10:1 N:P ratio corresponds to a 1.29:1 PEI:DNA weight ratio. 9NC-dilauroylphosphatidylcholine liposomes are prepared as described in ref 10.

### EXAMPLE 2

In Vitro Effect of p53 Gene and 9NC on B16-F10 Cell Proliferation

B16-F10 cells grown in tissue culture plates (20,000 cells/well in a 48 well plate; 1×10 cells/well in a 12 well plate) are transfected with PEI-DNA complexes (1 μg/ml; p53 or control empty plasmid, Neo) for 24 hours. The cultures were then washed with PBS and the transfection media was replaced with fresh media containing 9-nitrocamptothecin-dilauroylphosphatidylcholine (100 nM). The proliferation of B16-F10 cells 24 hours later was evaluated using the hematocytometer. To count the cells, the cell layer was washed with PBS and then trypsinized with 0.25% trypsin. The Alamar Blue Assay (Trek Diagnostic Systems, Inc) was also performed to test the viability of the cells (data not shown).

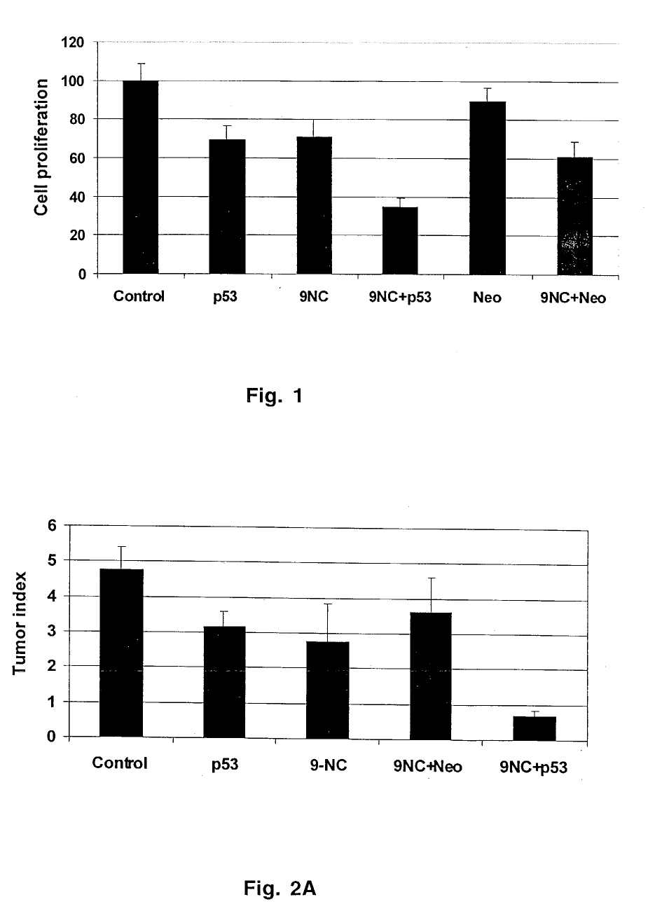

Control groups included cells treated with the genes (p53 and Neobam) alone, or with the drug (9-nitrocamptothecin) alone, or with Neobam and 9-nitrocamptothecin in combination. A very significant reduction in the proliferation of B16-F10 cells transfected with polyethylenimine-p53 and treated with 9-nitrocamptothecin-dilauroylphosphatidylcholine, as compared to cells treated with single agent alone or with polyethylenimine-Neobam and 9-nitrocamptothecin-dilauroylphosphatidylcholine combination demonstrated (FIG. 1). Furthermore, increased concentration of the drug (>0.5 mg/ml) or p53 plasmid (>2 μg/ml) showed very potent inhibition of B16-F10 inhibition as single agents alone, and did not reveal any additive effect when tested together (data not shown; 8). The p53 expression levels in parental and p53 treated B16-F10 cells have been reported previously (8). 9-nitrocamptothecin +p53 values are significantly different from the other groups (p<0.01; Students t test, two-tailed).

### EXAMPLE 3

In Vivo Effect of p53 Gene and 9NC on B16-F10 Metastases

C57BL/6 mice (7-8 weeks old, Harlan Sprague Dawley, Houston, Tex.) were injected via the tail vein with 25,000 B16-F10 cells on day 0. Starting on day 11 after tumor cell injection, when the tumor foci are well established in the lungs (not shown) and had reached microscopic size, the mice were treated twice a week with 9-nitrocamptothecin-dilauroylphosphatidylcholine and once a week with PEI-p53 aerosol complexes. Similar to in vitro studies, the p53 gene was delivered before the drug therapy; for combination therapy each week, the p53 was administered on Monday and 9-nitrocamptothecin was aerosolized on Tuesday and Friday. Animals in the single agent therapy group were administered p53 one time weekly or 9-nitrocamptothecin twice weekly.

The dosage for treatment was 2 mg plasmid/10 ml of aerosolized solution at a PEI:DNA (N:P) ratio of 10:1 for DNA, and 5 mg 9-nitrocamptothecin at a concentration of 0.5 mg/ml at a 9 nitrocamptothecin:dilauroylphosphatidylcholine weight ratio of 1:50. Aerosol delivery of drugs and genes was performed using 5% CO₂-in-air for increased pulmonary deposition. On day 25-post tumor inoculation, the mice were sacrificed, lungs were fixed and the tumor index was calculated.

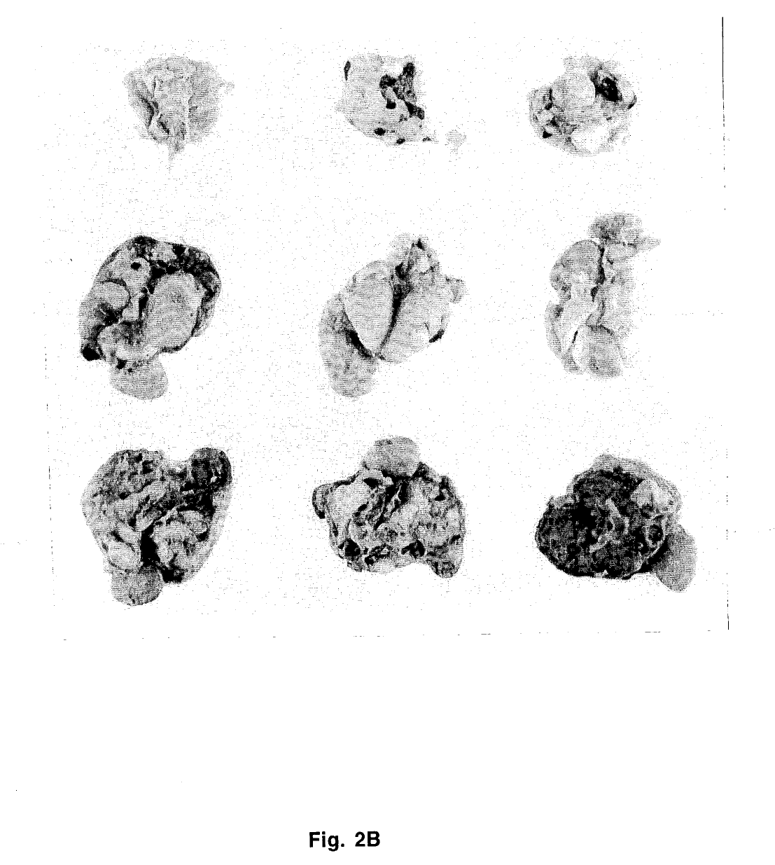

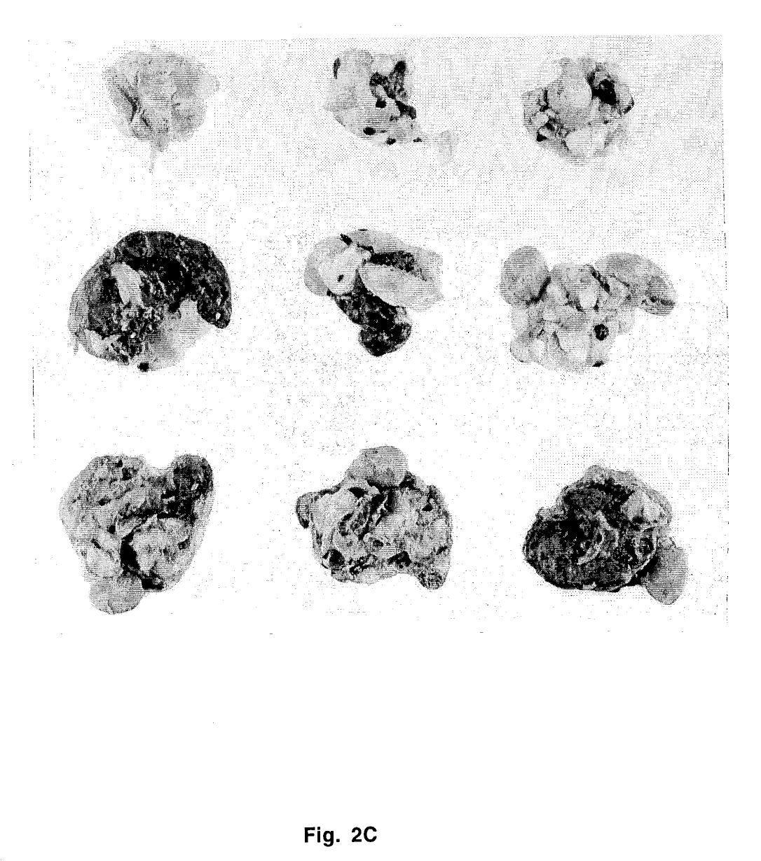

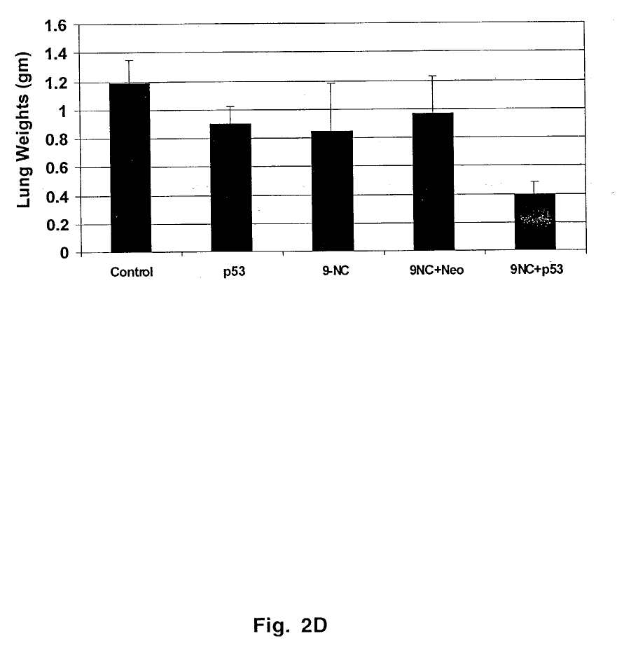

As shown in FIG. 2A, the mice treated with t h e combination of p53 and 9-nitrocamptothecin had significantly lower tumor index (p<0.001) compared to all other groups, whereas animals in all the other groups had large number of tumor nodules. All animals in the p53 and 9-nitrocamptothecin combination group had very small and distinct tumor foci (FIGS. 2B, 2C). The lungs from the 9-nitrocamptothecin-dilauroylphosphatidylcholine treated animals (not shown) were similar in size and shape to those shown for 9-nitrocamptothecin and PEI-Neobam combination. There was no effect of 5% CO₂ alone on the growth of tumors as compared to untreated mice (data not shown).

Tumor index was calculated by the formula: Tumor index=lung weights×average grade for the group. Since most of the lungs in the control groups had numerous uncountable foci, the lungs were graded based on a scale of 1-5 as previously described (8); 1 if there are less than 10 tumor foci, 2 if there are 10-100 tumor foci, 3 if one lobe of the lung is full of tumor, 4 if both lobes are full of tumor, and 5 if the lungs are full of tumor and the tumor was growing out of the lungs and into the chest wall. The growth of tumor contiguous to the lung was considered as a part of the lung for weighing the lungs. Values are mean+SD (n=10 mice per group).

The lung weights also revealed a significant difference between the 9NC+p53 combination group and all the other groups (p<0.01; Students t test, two-tailed) (FIG. 2D). The lung weight of sham-inoculated (no tumor) mice is about 0.17 gm.

### EXAMPLE 4

Survival of B16-F10 Challenged Mice with PEI-p53/9NC-DLPC Treatment

C57BL/6 mice (n=10 mice per group) were injected via the tail vein with 25,000 B16-F10 cells on day 0 and the tumor was allowed to establish until day 10. The mice were then left untreated; or exposed to PEI-p53 aerosol complexes, to 9-nitrocamptothecin alone or to a sequential combination of p53 and 9-nitrocamptothecin, or 9-nitrocamptothecin and Neobam combination starting day 11 after tumor cell injection. Neobam was used as a negative control. The dosage was twice a week for drug or gene alone; and in combination once a week for gene and, twice a week for drug therapy. The mice were treated for two weeks, and then monitored over time for their survival.

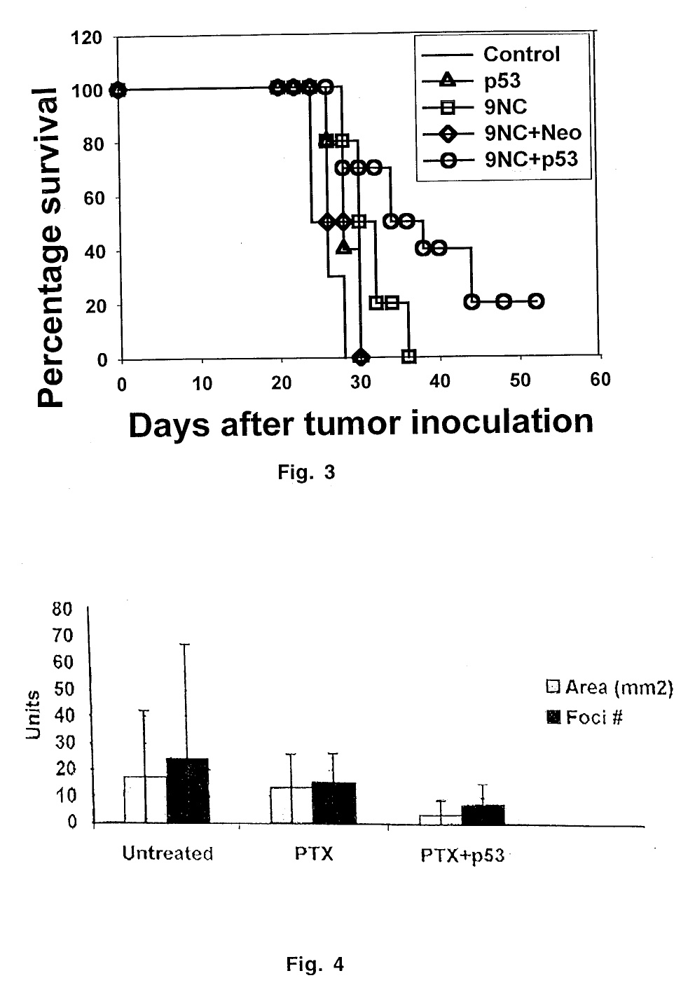

As shown in FIG. 3, the mean survival time of the mice treated with both p53 and 9-nitrocamptothecin was increased by 3040%, as compared to animals in other groups (37±6 days for 9NC+p53 as compared to 29±3 days for 9-nitrocamptothecin alone and 25÷4 days for other groups). The treatment was well tolerated. Furthermore, about 20% of mice in the p53 and 9-nitrocamptothecin combination group survived till day 50-post tumor inoculation and were tumor free (no visible tumor lesions on the lungs) when autopsied on day 52. Each step represents the number of mice dead at the indicated time point. Data is representative of at least 2 separate experiments. 9-nitrocamptothecin +p53 values are significantly different from the other groups (p<0.05; Mann-Whitney rank sum test).

### EXAMPLE 5

In Vivo Effect of p53 Gene and 9NC on Human SAOS LM-6 Osteosarcoma

It is contemplated that p53 might sensitize tumor cells to other anti-cancer drugs such as paclitaxel, as well as to irradiation therapy. The efficacy of paclitaxel (PTX) in combination with p53 therapy is examined in a human SAOS LM-6 osteosarcoma xenograft mouse model. 1×10⁶ SAOS LM-6 cells intravenously injected into the tail vein of immunodeficient (nu/nu) mice form pulmonary metastases in the animals. The tumors were allowed to grow for 8 weeks until they were micrometastases. The treatment was given according to the schedule described above; duration of treatment was 8 weeks. The mice were then sacrificed and tumors on the lung surfaces were counted and measured.

Antitumor effect was estimated by differences in lung weights between the treated and untreated group. Lungs with less tumor growth due to treatment weighed less than lungs from untreated mice with uninhibited tumor growth (FIG. 4). It appeared that paclitaxel combined with p53 gene treatment showed tumor inhibition was greater than an additive effect. P53 alone and paclitaxel alone had similar therapeutic effects (data not shown).

The following references are cited herein:

1. Lowe S W, Bodis S, McClatchey A, et al. P53 status and the efficacy of cancer therapy in vivo. Science 1994; 266:807-810.

2. Fujiwara T, Grimm E A, Mukhopadhyay T, et al. A retroviral wild-type p53 expression vector penetrates human lung cancer spheroids and inhibits growth by inducing apoptosis. Cancer Res 1993; 53(18):4129-33.

3. Horio Y, Hasegawa Y, Sekido Y, et al. Synergistic effects of adenovirus expressing wild-type p53 on chemosensitivity of non-small cell lung cancer cells. Cancer Gene Ther 2000; 7(4):537-44.

4. Koshkina N V, Gilbert B E, Waldrep J C, et al. Distribution of camptothecin after delivery as a liposome aerosol or following intramuscular injection in mice. Cancer Chemother Pharmacol 1999; 44(3):187-92.

5. Thierry A R, Lunardi-Iskandar Y, Bryant J L, et al. Systemic gene therapy: biodistribution and long-term expression of a transgene in mice. Proc Natl Acad Sci 1995; 92(21):9742-6.

6. Gautam A, Densmore C L, Xu B, and Waldrep J C. Enhanced gene expression in mouse lung after PEI-DNA aerosol delivery. Mol Ther 2000; 2(1):63-70.

7. Gautam A, Densmore C L, and Waldrep J C. Pulmonary cytokine responses associated with PEI-DNA aerosol delivery. Gene Ther 2001; 8:254-257.

8. Gautam A, Densmore C. L, and Waldrep J C. Inhibition of experimental lung metastasis by aerosol delivery of PEI-p53 complexes. Mol Ther 2000; 2(4):318-323.

9. Densmore C L, Kleinerman E, Gautam A, et al. Growth inhibition of established human osteosarcoma lung metastases in mice by aerosol delivery of PEI-p53 complexes. Cancer Gene Ther 2001; 8(9):619-627.

10. Koshkina N V, Kleinerman E S, Waldrep J C, et al. 9-Nitrocamptothecin liposome aerosol treatment of melanoma and osteosarcoma lung metastases in mice. Clin Cancer Res 2000; 6(7):2876-80.

11. Knight V, Koshkina N V, Waldrep J C, et al. Anticancer effect of 9-nitrocamptothecin liposome aerosol on human cancer xenografts in nude mice. Cancer Chemother. Pharmacol. 1999; 44(3): 177-86

12. Koshkina N V, Knight V, Gilbert B E, et al. Improved respiratory delivery of the anticancer drugs, camptothecin and paclitaxel, with 5% CO₂-enriched air: pharmacokinetic studies. Cancer Chemother Pharmacol. 2001; 47:451-456.

13. Gautam A, Densmore C L, Melton S, et al. Aerosol delivery of PEI-p53 complexes inhibits B16-F10 lung metastases through regulation of angiogenesis. Cancer Gene Therapy, 2001; In press.

14. Murren J R, Anderson S, Fedele J, et al. Dose-escalation and pharmacodynamic study of topotecan in combination with cyclophosphamide in patients with refractory cancer. J Clin Oncol 1997; 15(1):148-57.

15. Lebedeva S, Bagdasarova S, Tyler T, et al. Tumor suppression and therapy sensitization of localized and metastatic breast cancer by adenovirus p53. Hum Gene Ther 2001; 12(7):763-72.

16. Cummings J, Smyth J F. DNA topoisomerase I and II as targets for rational design of new anticancer drugs. Ann Oncol 1993; 4(7):533-43.

17. Liu W, Zhang R. Upregulation of p21WAF1/CIP1 in human breast cancer cell lines MCF-7 and MDA-MB-468 undergoing apoptosis induced by natural product anticancer drugs 10-hydroxycamptothecin and camptothecin through p53-dependent and independent pathways. Int J Oncol 1998; 12(4):793-804.

18. Nieves-Neira W, Pommier Y. Apoptotic response to camptothecin and 7-hydroxystaurosporine (UCN-01) in the 8 human breast cancer cell lines of the NCI Anticancer Drug Screen: multifactorial relationships with topoisomerase I, protein kinase C, Bcl-2, p53, MDM-2 and caspase pathways. Int J Cancer 1999; 82(3): 396-404.

19. Kalechman Y, Strassman G. Albek M, and Sredni B. Up-regulation by ammonium trichloro(dioxoethylene-0,0′) tellurate (AS101) of Fas/Apo-1 expression on B16 melanoma cells: implications for the antitumor effects of AS101. J. Immunol 1998; 161(7):3536-42.

20. Hanrahan D, and Folkman J. Patterns and emerging mechanisms of the angiogenic switch during tumorigenesis. Cell 1996; 86(3):353-64.

21. Clements M K, Jones C B, Cumming M, Daoud S S. Antiangiogenic potential of camptothecin and topotecan. Cancer Chemother Pharmacol 1999; 44(5):411-6.

22. O'Leary J J, Shapiro R L, Ren C J, et al. Antiangiogenic effects of camptothecin analogues 9-amino-20(S)-camptothecin, topotecan, and CPT-11 studied in the mouse cornea model. Clin Cancer Res 1999; 5(1):181-7.

23. Zhang L, Yu D, Hu M, et al. Wild-type p53 suppresses angiogenesis in human leiomyosarcoma and synovial sarcoma by transcriptional suppression of vascular endothelial growth factor expression. Cancer Res 2000; 60(13):3655-61.

24. Dameron K M, Volpert O V, Tainsky M A, Bouck N. Control of angiogenesis in fibroblasts by p53 regulation of thrombospondin-1. Science 1994; 265(5178):1582-4.

25. Fujiwara T, Grimm E A, Mukhopadhyay T, et al. Induction of chemosensitivity in human lung cancer cells in vivo by adenovirus-mediated transfer of the wild-type p53 gene. Cancer Res 1994; 54(9):2287-91.

26. Nemunaitis J, Swisher S G, Timmons T, et al. Adenovirus-mediated p53 gene transfer in sequence with cisplatin to tumors of patients with non-small-cell lung cancer. Journal of Clinical Oncology 2000; 18(3): 609.

27. Swisher S G, Roth J A, Nemunaitis J, et al. Adenovirus-mediated p53 gene transfer in advanced non-small-cell lung cancer. J Natl Cancer Inst 1999; 91(9):763-71.

28. Knight, V., et al. Cancer Chemotherapy Pharmacology 44:177-186 (1999))

Any patents or publications mentioned in this specification are indicative of the levels of those skilled in the art to which the invention pertains. Further, these patents and publications are incorporated by reference herein to the same extent as if each individual publication was incorporated specifically and individually by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of inhibiting growth of lung metastases in an individual comprising the steps of:
administering in a combination an aerosolized polyethylenimine-nucleic acid complex and an aerosolized liposome-anticancer drug complex;
wherein delivery of both of said nucleic acid and said anticancer drug inhibits growth of lung metastases in said individual.

2. The method of claim 1, wherein the combination comprises the steps of:
administering the aerosolized polyethylenimine-nucleic acid complex; and simultaneously
administering the aerosolized liposome-anticancer drug complex.

3. The method of claim 1, wherein the combination comprises the steps of:
administering the aerosolized polyethylenimine-nucleic acid complex; and sequentially
administering the aerosolized liposome-anticancer drug complex.

4. The method of claim 3, wherein the combination comprises a further step of:
readministering the aerosolized liposome-anticancer drug complex at least once.

5. The method of claim 1, wherein the administration of the combination of said aerosolized polyethylenimine-nucleic acid complex and said aerosolized liposome-anticancer drug complex is repeated at least once.

6. The method of claim 1 wherein said nucleic acid is selected from the group consisting of DNA, RNA, a catalytically active nucleic acid, a ribozyme, an antisense oligonucleotide, and a modified nucleic acid.

7. The method of claim 6, wherein said nucleic acid exhibits tumor suppressor activity.

8. The method of claim 7, wherein said nucleic acid is a DNA selected from the group consisting of the p53 gene, a truncated derivative of the p53 gene, the CD1 gene, interleukin-12, and interferon-γ.

9. The method of claim 1, wherein said anti-cancer drug is selected from the group consisting of 9-nitrocamptothecin, paclitaxel, doxorubicin, carboplatin, methotrexate, vinblastine, etoposide, docetaxel hydroxyurea, fluorouracil, busulfan, imatinib mesylate, alembuzumab, aldesleukin, and cyclophosphamide.

10. The method of claim 1, wherein said liposome is dilauroylphosphatidylcholine.

11. The method of claim 1, wherein said aerosol comprises about 3% to about 7.5% carbon dioxide.

12. The method of claim 1, wherein a ratio of polyethylenimine nitrogen to nucleic acid phosphate is about 2:1 to about 50:1.

13. The method of claim 12, wherein said ratio of polyethylenimine nitrogen to nucleic acid phosphate is about 5:1 to about 20:1.

14. The method of claim 1, wherein said polyethylenimine-nucleic acid complex is polyethylenimine-p53 and said liposome-anticancer drug complex is dilauroylphosphatidylcholine-9-nitrocamptothecin.

15. The method of claim 14, wherein said p53 is administered in a dose comprising about 2 mg p53 plasmid/10 ml of aerosolized solution.

16. The method of claim 14, wherein said 9-nitrocamptothecin is administered in a dose comprising about 0.5 mg 9-nitrocamptothecin/ml at a 9-nitrocamptothecin:dilauroylphosphatidylcholine weight ratio of about 1:50.

17. A method of inhibiting growth of lung metastases in an individual comprising the steps of:
administering in a combination an aerosolized polyethylenimine-p53 complex and an aerosolized dilauroylphosphatidylcholine-9-nitrocamptothecin complex;
wherein delivery of both p53 and 9-nitrocamptothecin inhibits growth of lung metastases in said individual.

18. The method of claim 17, wherein the combination comprises the steps of:
administering the aerosolized polyethylenimine-p53 complex; and simultaneously
administering the aerosolized dilauroylphosphatidylcholine-9-nitrocamptothecin complex.

19. The method of claim 17, wherein the combination comprises the steps of:
administering the aerosolized polyethylenimine-p53 complex; and sequentially
administering the aerosolized dilauroylphosphatidylcholine-9-nitrocamptothecin complex.

20. The method of claim 19, wherein the combination comprises a further step of:
readministering the aerosolized dilauroylphosphatidylcholine-9-nitrocamptothecin complex at least once.

21. The method of claim 17, wherein the administration of the combination of said aerosolized polyethylenime-p53 complex and said aerosolized dilauroylphosphatidylcholine-9-nitrocamptothecin complex is repeated at least once.

22. The method of claim 17, wherein a ratio of polyethylenimine nitrogen to p53 phosphate is about 5:1 to about 20:1.

23. The method of claim 22, wherein said ratio of polyethylenimine nitrogen to p53 phosphate is about 10:1.

24. The method of claim 17, wherein said p53 is administered in a dose comprising 2 mg p53 plasmid/10 ml of aerosolized solution.

25. The method of claim 17, wherein said 9-nitrocamptothecin is administered in a dose comprising 0.5 mg 9-nitrocamptothecin/ml at a 9-nitrocamptothecin:dilauroylphosphatidylcholine weight ratio of 1:50.

26. The method of claim 17, wherein said aerosol comprises about 3% to about 7.5% carbon dioxide.

27. The method of claim 26, wherein said aerosol comprises about 5% carbon dioxide.
